(19) United States
(12) Patent Application Publication
(10) Pub. No.: US 2025/0058085 A1
(43) Pub. Date: Feb. 20, 2025

(54) Systems And Methods For Automated Peripheral Vessel Catheterization

(71) Applicant: Rutgers, The State University of New Jersey, New Brunswick, NJ (US)

(72) Inventors: Martin L. Yarmush, Newton, MA (US); Alvin Chen, Cambridge, MA (US); Max Balter, Boston, MA (US); Joshua Leipheimer, Highland Park, NJ (US)

(73) Assignee: Rutgers, The State University of New Jersey, New Brunswick, NJ (US)

(21) Appl. No.: 18/723,515

(22) PCT Filed: Dec. 5, 2022
(86) PCT No.: PCT/US2022/051803

§ 371 Date: Jun. 24, 2024

Related U.S. Application Data
(60) Provisional application No. 63/293,406, filed on Dec. 23, 2021.

(51) Int. Cl. A61M 25/01; A61B 17/34; A61B 34/32; A61M 5/42; A61M 25/06; G06T 7/00; G16H 40/63

(52) U.S. Cl. CPC A61M 25/0113; A61M 5/427; A61M 25/0693; G06T 7/0012; G16H 40/63; A61B 17/3403; A61B 34/32; A61M 2205/0272; A61M 2205/3313; A61M 2205/332; A61M 2205/3368; A61M 2210/12; G06T 2207/30021; G06T 2207/30101

(57) ABSTRACT

A device for catheterization includes a catheter sheath support mounted to a catheterization device and holding a catheter sheath, a carriage supporting a guide needle, and a connector to removably engage the catheter sheath support to the carriage. The carriage translates along an axis of insertion of the guide needle independent of the catheter sheath support when disengaged from the connector. An actuator controls separation of the carriage from the catheter sheath support at a uniform velocity in response to disengagement of the connector. Translation and separation may be automated. In response to detection of the vessel wall puncture, the carriage automatically releases from the catheter sheath support and retracts along the axis of insertion at the uniform velocity relative to the catheter sheath support, and the catheter sheath automatically advances into the target vessel along the axis of insertion at the uniform velocity while the carriage retracts.

### CROSS-REFERENCE TO RELATED APPLICATIONS

The present application claims the benefit of the filing date of U.S. Provisional Application No. 63/293,406, filed Dec. 23, 2021, entitled “Systems And Methods For Automated Peripheral Vessel Catheterization.” the disclosure of which is hereby incorporated herein by reference.

### STATEMENT REGARDING FEDERALLY SPONSORED RESEARCH OR DEVELOPMENT

This invention was made with government support under R01EB020036 and F31HL149219 awarded by the National Institutes of Health. The government has certain rights in the invention.

### BACKGROUND OF THE INVENTION

Percutaneous peripheral catheterizations are typically performed manually by trained practitioners. The practitioner visually locates or palpates for a tissue target such as a blood vessel and then introduces a cannula aiming to reach the center of the target, often in a blind manner. Commonly, it is difficult to find a suitable target, particularly in young children, elderly or obese patients, or patients with comorbidities. It may also be difficult to estimate the depth or to insert the instrument accurately in the presence of tissue motion. For these reasons, successful cannulation depends heavily on the patient's physiology and the practitioner's skill. For vascular access in particular, failure rates are known to exceed 25% overall and can increase up to 70% in challenging patients. Difficulties in finding the vessel, or in inserting the needle or catheter, increase the likelihood of pain, bruising, and access-related complications. Such difficulties also lead to significant delays to treatment and result in unnecessary costs to the health care facility.

Control schemes for the handheld automated cannulation devices are capable of quickly and efficiently responding to real-time sensor data while not having to rely on a fixed frame of reference external to the device itself. While the known control schemes are capable of enabling a practitioner to perform cannulation for routine blood draw venipuncture, they are not sufficient for performing peripheral catheterizations for intravenous or intravascular therapy. Peripheral catheterizations involve guiding a catheter sheath and guide needle into a blood vessel and retracting the guide needle out post-puncture, while the catheter sheath remains in place. This is more involved than merely puncturing the vessel and drawing blood.

### BRIEF SUMMARY OF THE INVENTION

The present disclosure is directed to a handheld automated device that is capable of performing percutaneous peripheral catheterizations, as well as blood draws. The device combines the imaging, positioning, and continuous image-based feedback technologies of known control schemes for handheld automated cannulation devices with additional features to safely guide a catheter sheath and guide needle into a blood vessel and retract the guide needle out post-puncture while the catheter sheath remains in place. The additional features may include both hardware components for steadying the catheter sheath and guide needle during puncture and withdrawal, as well as routines for steady control of the hardware components.

One aspect of the present disclosure is directed to an automated cannulation method may include: receiving, by one or more processors, imaging data of a target location containing one or more target vessels for insertion of a cannula under a patient's skin; identifying, by one or more processors, a plurality of candidate vessel segments from among the one or more vessels; determining, by the one or more processors, a plurality of characteristics of each of the candidate vessel segments based on the imaging data, wherein at least one of the plurality of characteristics is a vessel cross-sectional area; assigning, by the one or more processors, a respective plurality of values for each of the identified candidate vessel segments based on the determined plurality of characteristics, each value corresponding to a respective characteristic; calculating, by the one or more processors, a total score for each of the identified candidate vessel segments based on the respective plurality of values; selecting, by the one or more processors, a highest scoring candidate vessel segment based on the calculated total scores; and outputting, by the one or more processors, the selected highest scoring candidate vessel segment.

In some examples, the method may further include: assigning a respective predetermined weight to each of the respective plurality of values of the plurality of identified candidate vessel segments. For each identified candidate vessel segment, the total score of the identified candidate vessel segment may be a weighted sum of its corresponding plurality of values.

In some examples, the plurality of characteristics may include a distance of the identified candidate vessel segment from the patient's arteries. The distance of the identified candidate vessel segment from the patient's arteries may be determined based on the imaging data.

In some examples, the plurality of characteristics may include a quality of blood flow through the identified candidate vessel segment. The quality of blood flow may be determined based on doppler signal strength derived from the imaging data.

In some examples, identifying the plurality of candidate vessel segments from among the one or more vessels may be performed using a machine learning model. The plurality of characteristics may include a confidence level output by the machine learning model. In some examples, the machine learning model may be a convolutional neural network.

Another aspect of the present disclosure is directed to a method for automated catheterization including: detecting, by a sensor, vessel wall puncture of a target vessel by a needle supporting a catheter sheath; and in response to detection of the vessel wall puncture: automatically releasing a carriage of the needle from a catheter sheath support to which the catheter sheath is mounted; automatically retracting the carriage along an axis of insertion of the needle at a uniform velocity relative to the catheter sheath support; and automatically advancing the catheter sheath into the target vessel along the axis of insertion of the needle at the uniform velocity while the carriage is automatically retracting such that a position of the needle relative to the target vessel is maintained.

In some examples, automatically retracting the carriage may involve applying a constant force to the carriage in a direction away from the target vessel along the axis of insertion of the needle. The constant force may be controlled to not exceed a predetermined threshold.

In some examples, the sensor may be a force sensor. Automatically releasing the carriage may involve actuating a solenoid latch in response to detection of the vessel wall puncture by the force sensor.

In some examples, the method may further include receiving an indication of successful catheterization separate from the indication of vessel wall puncture. Automatically releasing the carriage may be performed in further in response to both the detection of vessel wall puncture and the indication of successful catheterization.

In some examples, the indication of successful catheterization may be determined from at least one of: an infrared emitter and collector sensor for detecting blood flash; or a force profile of force data collected at the force sensor during insertion of the needle into the target vessel.

In some examples, the method may further include: receiving, by one or more processors, imaging data of a target location under a patient's skin; identifying, by the one or more processors, a plurality of candidate vessel segments from among the one or more vessels; assigning, by the one or more processors, a respective likelihood of success value for each of the identified candidate vessel segments, the likelihood of success value indicating a likelihood of success of canulation using the corresponding candidate vessel segment; determining, by the one or more processors, a cross-sectional area of each of the candidate vessel segments based on the imaging data; assigning, by the one or more processors, a respective size value for each of the identified candidate vessel segments; calculating, by the one or more processors, a total score for each of the identified candidate vessel segments based on the respective likelihood of success values and the respective size values; selecting, by the one or more processors, a highest scoring candidate vessel segment based on the calculated total scores; and advancing, by the one or more processors, the guide needle and catheter towards the selected highest scoring candidate vessel segment of the target vessel.

Yet another aspect of the present disclosure is directed to a device for automated catheterization including: a catheter sheath support configured to be mounted to a catheterization device, the catheter sheath support being configured to hold a catheter sheath; a carriage configured to support a guide needle; a connector configured to removably engage the catheter sheath support to the carriage, the carriage being configured to translate along an axis of insertion of the guide needle independent of the catheter sheath support when the carriage is disengaged from the connector; and a constant force actuator configured to control separation of the carriage from the catheter sheath support at a uniform velocity in response to disengagement of the connector.

In some examples, the constant force actuator may be a spring.

In some examples, the uniform velocity may be equal to a velocity of insertion of the catheter sheath controlled by an injection motor of the handheld catheterization device.

In some examples, the device may further include a rotary speed limiter configured to limit a magnitude of the uniform velocity to a predetermined threshold.

In some examples, the device may further include a force sensor positioned in line with the axis of insertion of the guide needle and is configured to: detect vessel wall puncture; and transmit a vessel wall puncture signal in response to detection of the vessel wall puncture.

In some examples, the connector may be configured to disengage in response to the vessel wall puncture signal.

In some examples, the connector may be a solenoid latch, and the vessel wall puncture signal may be an electrical voltage for actuating the solenoid latch.

In some examples, the device may further include a blood flash sensor configured to detect successful insertion of the guide needle. The connector may be configured to disengage further in response to a signal indicating detection of successful insertion of the guide needle by the blood flash sensor.

The devices and methods disclosed herein are advantageous for patients, practitioners and healthcare facilities. For patients, the device and workflow ensure rapid, single-attempt cannulation success, particularly in difficult-access patients. For practitioners, the device has the potential to eliminate the risk of accidental sharps injuries by automating the instrument loading and disposal processes. Finally, for healthcare facilities, the device has the potential to reduce costs due to complications and delays, and may potentially allow less trained personnel to perform procedures.

### BRIEF DESCRIPTION OF THE DRAWINGS

FIG. 1 is a side view of an example catheterization device in accordance with an aspect of the present disclosure.

FIG. 2 is a functional block diagram of the catheterization device of FIG. 1.

FIGS. 3 (a)-3(c) are perspective views of a portion of the catheterization device of FIG. 1 while retracting a guide needle from a catheter sheath after successful insertion into a target vessel.

FIG. 4 (a) is an additional perspective view of the catheterization device of FIG. 1.

FIG. 4 (b) is a side view of an example of the catheterization device of FIG. 1

FIGS. 5 (a)-5(c) are side views of another example of the catheterization device of FIG. 1.

FIGS. 6 (a)-6(b) are side views of yet another example of the catheterization device of FIG. 1

FIG. 7 is a block diagram of a workflow for operating a catheterization device in accordance with an aspect of the present disclosure.

FIG. 8 is a flow diagram of an automated catheterization protocol according to an aspect of the present disclosure.

FIG. 9 is a flow diagram of a target vessel selection protocol according to an aspect of the present disclosure.

### DETAILED DESCRIPTION

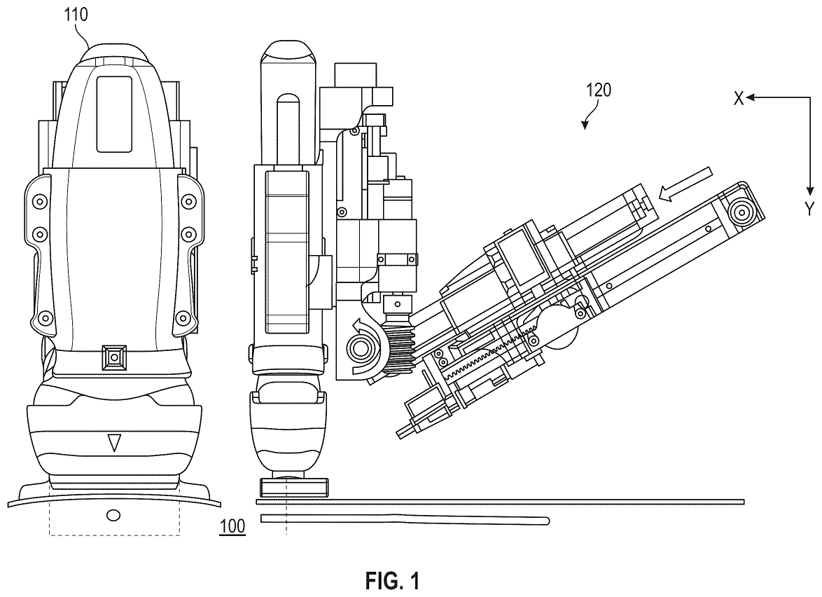

FIG. 1 illustrates an example catheterization device 100. The device 100 may be handheld and may be automated in order to improve accuracy of the catheterization process. The device 100 includes both an imaging unit 110 for imaging the target arca for catheterization and a positioning unit 120 for manipulating a needle towards the target area based at least in part on information from the imaging unit 110.

The imaging unit 110 may include an ultrasound probe for acquiring images of a target arca underneath the skin of the patient, such as one or more blood vessels. The probe may be attached to an end of the unit 110, whereby the unit 110 is configured to acquire an ultrasound (US) image of a portion of the patient positioned opposite the probe. The probe may be made from a flexible material designed to flex and conform to the imaged portion of the patient (e.g., the patient's arm). In some examples, the probe may further include a gel compartment configured to contain ultrasound gel for improving acquisition of the US image.

The positioning unit 120 may be attached to the imaging unit 110, and may include a robotic arm configured to control the positioning of a needle. In the case of the catheterization device, the robotic arm may further control the positioning of a catheter sheath. Each of the needle and catheter sheath may be supported by respective attachments to the positioning unit 120. In some instances, the attachments may be engaged with one another such that movement of the needle and catheter sheath is in unison. In other cases, the attachments may be disengaged so that each can move independently of the other, such as during disengagement of a guide needle from the catheter sheath after a successful puncture of a target vessel. The positioning unit 120 may be capable of movement along multiple degrees of freedom for manipulating the needle and catheter sheath, individually or in unison.

Example imaging units and positioning units are described in co-owned U.S. application Ser. No. 17/284,018, the disclosure of which is hereby incorporated in its entirety herein. For instance, near-infrared (NIR) light imaging may be used instead of or in addition to ultrasound imaging.

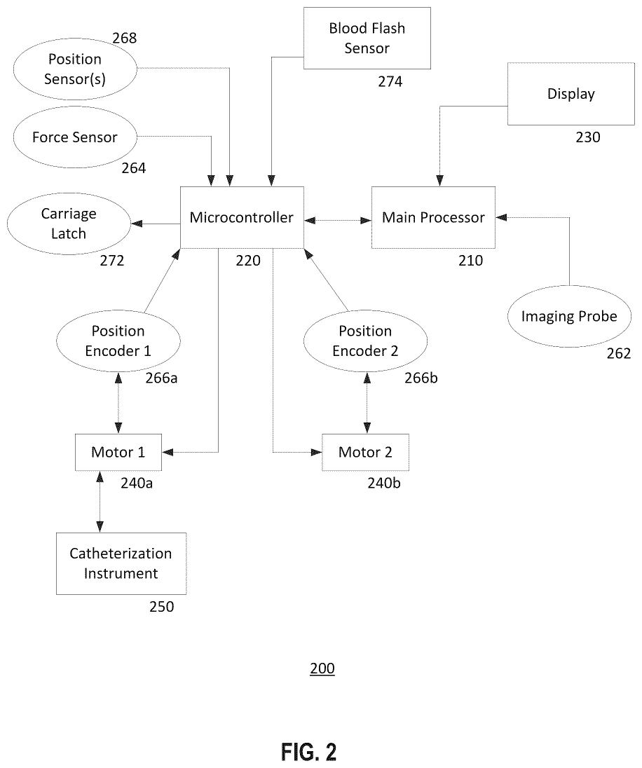

FIG. 2 is a block diagram illustrating a control scheme for a device 200 according to the present disclosure. The catheterization device 200 includes each of a main processor 210, a microcontroller 220, a display 230, and a plurality of motor controllers 240a, 240b for controlling various axes of freedom of a catheterization instrument 250, such as a guide needle, catheter sheath, or both. The main processor 210 may be any processing unit known in the art with sufficient processing power to receive process and output image data at video frame rates. The microcontroller 220 may be an integrated chip or other processing unit known in the art with sufficient processing capacity to control a robotic arm, including the processing and relaying of sensor inputs and instructions for controlling motor activity.

The microcontroller 220 may be configured to receive either one or a combination of analog and digital inputs from various sensors, to process those inputs, and relay the inputs to the main processor over a controller area network (CAN) connection. In some instances, the main processor 210 may include a different connection, such as a universal serial bus (USB) connection, in which case, the sensor data may be relayed over a CAN-to-USB connection. Conversely, instructions relayed from the main processor 210 through the microcontroller 220 may be provided through a USB-to-CAN connection.

The display 230 may be configured to provide a two-dimensional or three-dimensional image of the cannulation target location and the cannulation instrument. A cannulation trajectory, such as an expected trajectory of the cannulation instrument to reach the target vessel, may be superimposed on the image. The image may be updated or refreshed in real time or at video frame rates. The display may further be configured to allow a clinician to specify a location in an image presented thercon, such as specifying a target location.

The display 230 may further be configured to initialize image processing routines. For instance, automatic segmentation of the boundaries of a vessel may be initialized through the interaction between the user and the device 200 through the display 430. For instance, the ultrasound transducer may be moved around until the target vessel is centered in the ultrasound image (the display may include crosshairs to indicate the image center). Once centered, processing software in the device may be manually started to search for a segmentation starting from the center of the image. After the initial segmentation, the target can be tracked automatically afterwards.

The catheterization device 200 further includes a plurality of sensors for providing inputs to the main processor 210 and microcontroller 220. The sensors may include an imaging probe 262, such as an ultrasound probe, a force sensor 264 for sensing forces applied to the guide needle or to the motor as the positioning unit operates, respective position encoders 266a, 266b for each motor controller 240a, 240b to monitor manipulation of the needle by the device's motors, a position sensor 268 for tracking changes in translation and orientation of the handheld catheterization device 200, a carriage latch 272 for disengaging a guide needle carriage from a support of the catheter sheath, and a blood flash sensor 274 for detecting successful puncture or cannulation of a target vessel. For a catheterization device that is handheld, the position sensor 268 is necessary to track if the catheterization device is moved from its initial location, cither intentionally or inadvertently.

The main processor 210 and microprocessor 220 may be programmed with software instructions for carrying out the operations and protocols outlined in the present disclosure. For instance, the microprocessor may be programmed to receive analog or digital inputs from each of the force sensor, position encoders, position sensor, and blood flash sensor, process the received inputs into a format acceptable to the main processor, and then transmit the reformatted input data to the main processor for further processing. The microcontroller may further be responsible for controlling operation of various components of the device, such as the motors and the carriage latch, in order to control movement at the positioning unit. The main processor may be programmed to receive and process imaging data, such as identifying a target location in the patient and determining a trajectory for guiding the catheterization instrument to the target location. The determined trajectory may include velocities, accelerations, or both along various degrees of freedom controlled by the motors, such that operating the motors at the indicated velocities and/or accelerations may result in the catheterization instrument following a desired path to its target location. The main processor may further determine and continuously track a relative position of the catheterization instrument to the target location based on the received inputs, and may update the determined trajectory based on changes in the relative position.

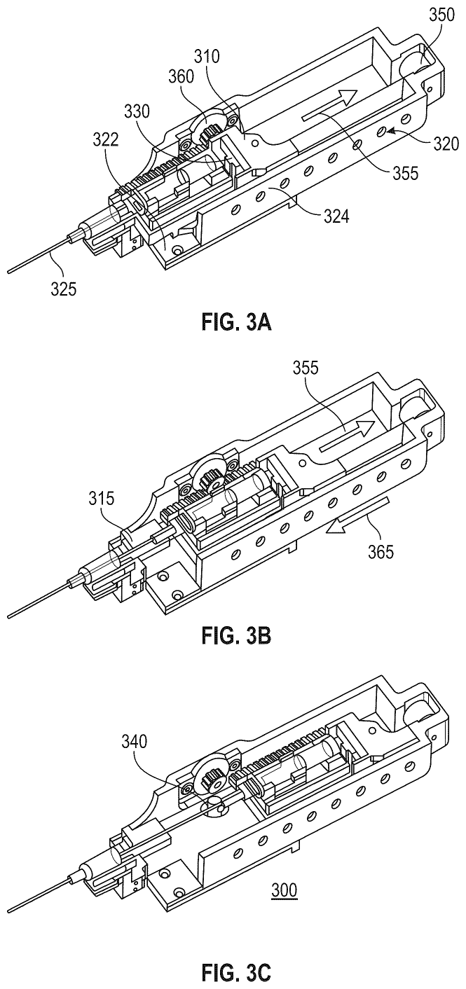

FIGS. 3A-3C illustrate a time lapse of a mechanical arrangement 300 for controlling the movement of a guide needle 315 and catheter sheath 325, independently or in unison. FIG. 3A illustrates an initial configuration of the mechanical arrangement 300, such as at the time when a successful puncture of the target vessel is detected. FIG. 3B illustrates a next configuration after the initial configuration, in which a carriage supporting the guide needle has disengaged from the push or support that is supporting the catheter sheath. Finally, FIG. 3C illustrates a final configuration at which the mechanical arrangement 300 may come to rest after the disengagement shown in FIG. 3B.

In the initial configuration, the guide needle 315 and catheter sheath 325 are connected to one another, such that the guide needle 315 provides structural support for the catheter sheath 325 as the sheath is inserted into the target vessel. A distal portion of the guide needle 315 is attached to a carriage 310. In the example of FIG. 3A, the carriage 310 is configured to receive the guide needle 315 through an opening, such that the guide needle may be removed after use. The catheter sheath 325 is supported by a catheter sheath support 320, such as a push. In the example of FIG. 3A, the catheter sheath support 320 includes a base 322 and sidewalls 324. The base 322 and sidewalls 324 may define a space receiving the carriage 310 of the guide needle 315. The carriage 310 may be configured to move along a lengthwise axis of the catheter sheath support 320. In the initial position, the carriage may be positioned at a front of the carriage, whereby the carriage may be capable of moving backward but not forward relative to the catheter sheath support 320.

The mechanical arrangement 300 may further include a connector 340. The connector 340 may be positioned between the carriage 310 and the catheter sheath support 320 and configured to maintain engagement, such as a locked or latched connection, between the carriage 310 and the catheter sheath support 320. In the example of FIG. 3A, the connector is positioned between a base of the carriage 310 and an upper surface of the base 322 of the catheter sheath support 320, and as such cannot be seen in the illustration. The connector 340 may be any one or combination of connection implements, including but not limited to latches locks, magnets, bolts, and so on. In the example of FIG. 3C, the connector 340 is shown as a solenoid, latch, or more particularly as a plunger that is controlled between a latching and a releasing position by a solenoid coil.

The mechanical arrangement may also include one or more sensors 330 for detecting puncture of the vessel wall by the guide needle 315 and catheter sheath 325. In the example of FIG. 3A, a force sensor 330 is included in the carriage 310 to detect forces applied to the guide needle. The force sensor 330 may be coupled to a distal end of the guide needle, whereby puncture of the vessel wall may be detected by a force applied to the force sensor. For instance, an increase in force followed by a sudden decrease may indicate successful puncture. The force sensor may be capable of detecting forced between about 0-5 N. Other sensors may be included in addition to or instead of the force sensor. For instance, a blood flash sensor may be provided to indicate successful catheterization. The blood flash sensor may include an infrared light emitter and sensor, whereby infrared light emitted by the emitter and reflected off blood that enters into the catheter may bounce back towards the infrared sensor to indicate the presence of blood in the catheter. Other sensors and techniques may include detecting puncture using a pressure sensor, determining puncture based on imaging, such as ultrasound or other image types received from the imaging unit, position tracking, such as with position encoders included with the motors of the positioning unit, temperature sensing such as with a thermocouple to detect blood contacting the needle tip, or a combination thereof.

In response to detection of a successful puncture of the vessel wall of the target vessel, the sensor 330 may automatically transmit a signal to the connector 340 to disengage the carriage 310 from the catheter sheath support 320. The carriage 310 may be biased by a constant force actuator 350 such as one or more springs configured to apply a constant force. The actuator may be attached to the catheter sheath support 320 such that the force cannot move the carriage 310 while the carriage 310 remains engaged with the catheter sheath support 320. However, once the carriage 310 is disengaged from the catheter sheath support 320, the biasing force of the actuator 350 may cause the carriage 310 to retract from a front position towards a back position of the space within the catheter sheath support 320, symbolized by arrow 355 in FIGS. 3A and 3B.

The constant force applied by the actuator 350 may cause the carriage to move at a uniform velocity relative to the catheter sheath support 320. At the same time, one or more motors of the positioning unit may continue to advance the guide needle and catheter sheath towards the target vessel in order to complete the catheterization process, as symbolized by arrow 365 in FIG. 3B. Advancing the guide needle 310 and catheter sheath 320 may also be performed at a uniform velocity, and particularly the same uniform velocity as retraction of the carriage. The uniform velocity may be between 2-15 mm/s. In some embodiments, a velocity greater than 15 mm/s may be used. In effect, from the frame of reference of the catheter sheath support 320, the carriage 310 and guide needle 315 may appear to translate backwards at a unform velocity. However, from the frame of reference of the target vessel, the carriage 310 and guide needle 315 may appear to remain stationary, while the catheter sheath support and catheter sheath advance further into the target vessel.

In some examples, a force of between about 0.4 lbs and 1 lb may be applied by the actuator 350. More specifically, a force of between about 0.6 and 0.7 lbs may be applied. The applied force may be further control by providing a force limiting or speed limiting element 360 to limit the force applied. In the example of FIG. 3A and 3B, a rotary speed limiter, which may be a rotary damper 360, attached to an inner surface of a sidewall 324 of the catheter sheath support 320 is shown. The rotary damper 360 may interact with the base of the carriage 310, thereby maintaining a limited velocity at which the carriage 310 may retract. This prevents buckling at the catheter sheath from occurring during the guide needle removal process. In other examples, other speed limiting elements may be used, including but not limited to friction tape, a constant-force rotary spring, or another motor.

Finally, in FIG. 3C, the guide needle 315 is shown as having fully separated from the catheter sheath 325. In the example of FIG. 3C, this involves the guide needle reaching a backwall of the catheter sheath support 320 in which the actuator 350 is positioned. At this stage, the guide needle 315 can be easily removed and disposed of without disturbing the catheter.

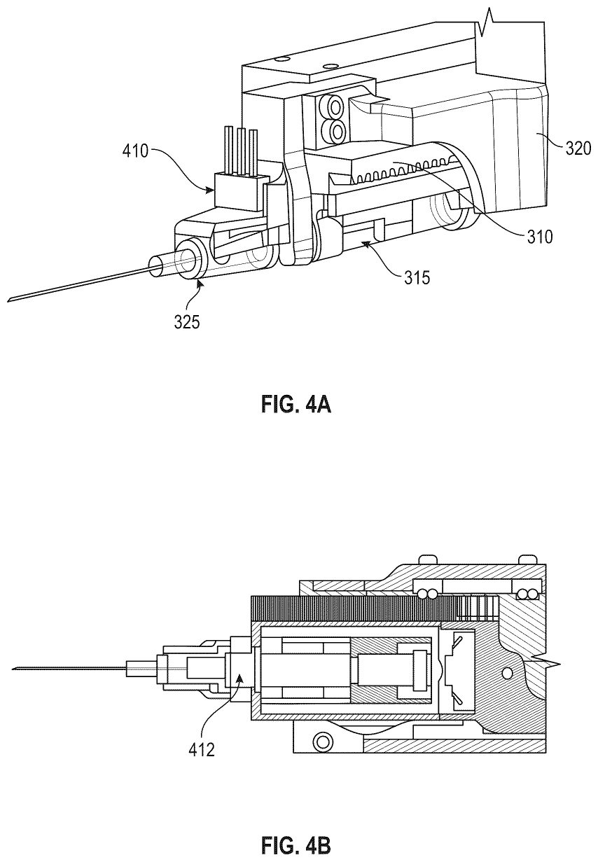

Additional features of the mechanical arrangement can be observed from the upside-down perspective view and sideview images shown in FIG. 4. For example, the blood flash sensor 410 can be seen to be positioned on a surface of the catheter sheath support 320 just underneath the catheter sheath 325. Also shown in FIG. 4, the guide needle may include a blood flash chamber 412 for collecting blood from the catheter sheath 325.

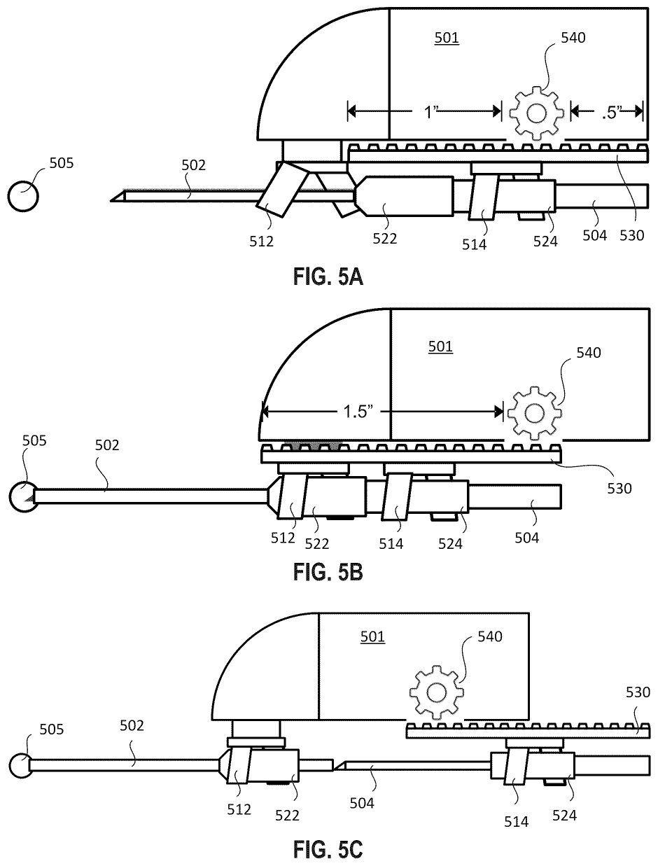

In the example arrangement of FIGS. 3 and 4, the guide needle and catheter sheath are supported by a carriage. However, in other arrangements, the guide needle and catheter sheath may be held in place and manipulated using other holding mechanisms. For instance, in the example catheterization device 501 of FIGS. 5A-5C, the catheter sheath 502 and the guide needle 504 and are held in place by a first gripper 512 and a second gripper 514, respectively. Each of the grippers 512, 514 may include two or more arms or prongs extending from a bottom surface of the body of the catheterization device 501. An angle formed by the arms of the first gripper 512 may be controllable between a closed position in which the arms are held at a relatively narrow angle with one another and the catheter sheath 502 is held in place by the arms, and an open position in which the arms are held at a relatively wide angle to one another and the catheter sheath 502 is free to move. Similarly, the arms of the second gripper 514 may be controllable between a closed position in which the guide needle 504 is held in place and an open position in which the guide needle 504 is free to be released from the second gripper 514. In order to facilitate a grip on the guide needle 502 and the catheter sheath 504, each of the catheter sheath 502 and guide needle 504 may include a respective casing 522, 524 having a width wider than the catheter sheath 502 and guide needle 504, respectively. The first and second grippers 512, 514 may be adapted to grip objects having a width approximately equal to a diameter of the respective casings 522, 524.

The first gripper 512 may be fixed in place relative to the bottom surface of the body of the catheterization device 501. The second gripper 514 may be mounted to a track 530 capable of translating forward and backward relative to the body of the catheterization device 501, whereby forward is a direction towards the target vessel 505 and backward is an opposite direction away from the target vessel 505. As such, when the catheter sheath 502 and guide needle 504 are connected to one another and are collectively held by the second gripper 514, the catheter sheath 502 and guide needle 504 may be advanced toward the target vessel 505 by actuating the track in a forward direction. Conversely, when the catheter sheath 502 is gripped by the first gripper 512, then the catheter sheath 502 may be prevented from moving forward or backward relative to the body of the catheterization device 501.

In operation, and as shown in FIG. 5A, the catheter sheath 502 and guide needle 504 may begin as being gripped by the second gripper 514 and aligned with the target vessel 505. In the initial position, the rail may include about a half inch of track for advancing the guide needle 504. Next, and as shown in FIG. 5B, a motor may cause the track 530 to move, such as by rotating a gear 540 coupled to the track 530 so as to shift objects positioned on the track 530 relative to the body of the catheterization device 501, thereby advancing the catheter sheath 502 and guide needle 504 towards the target vessel 505. The advance may be approximately a half inch in distance. In response to reaching the target vessel, which may be sensed using any of the example sensing mechanisms described in the other examples of the present disclosure, the first gripper 512 may transition from the open state to the closed state, thereby gripping the casing 522 of the catheter sheath 502. During this time the second gripper 514 may maintain its grip on the guide needle 504. Next, and as shown in FIG. 5C, the motor may rotate the gear 540 in an opposite direction so as to shift the track 530 relative to the body of the catheterization device 501 in an opposite direction, thereby moving the guide needle 504 which is still gripped by the second gripper 514 away from the target vessel 505. However, since the catheter sheath 502 is engaged with the first gripper 512, the catheter sheath 502 may remain in place and not move back with the guide needle 504, thereby separating the guide needle 504 from the catheter sheath 502. In the example of FIG. 5C. the backward translation motion of the guide needle 504 is shown to be about 1.5 inches.

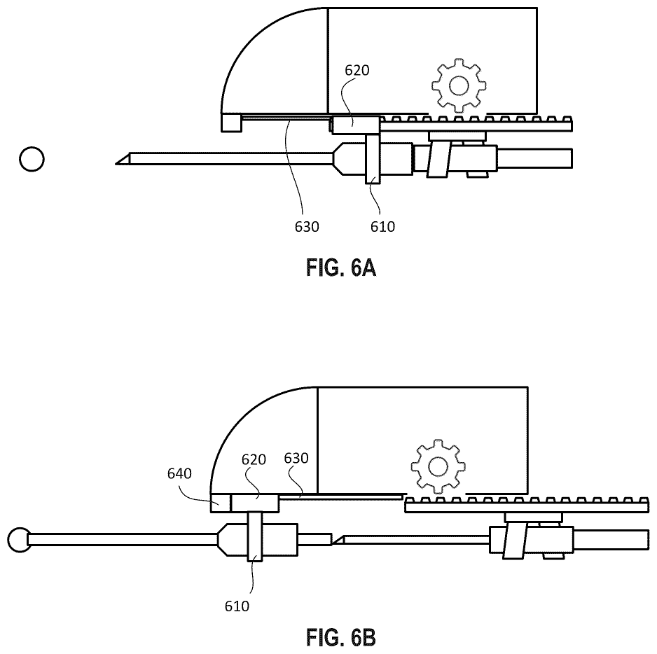

In the example of FIGS. 5A-5C, individually controllable mechanisms are required for controlling respective grips on the catheter sheath and guide needle. Alternatively, no control mechanism may be required for the second gripper 514, since the second gripper 514 may remain engaged to the guide needle at all times. For example, the second gripper 514 may be any attachment mechanism for releasably connecting a catheter sheath to the device, including but not limited to a clip, a fastener, and so on. In yet further arrangements, it may be possible to control motion of the catheter sheath without transitioning an arm between open and closed positions. For instance, in the example of FIGS. 6A and 6B, the catheter sheath is shown to be held by a support having one or more arms 610 extending from a bottom surface of the body of the catheterization device. The one or more arms may have a single configuration for holding the catheter sheath, and may not be configured to open and close. The one or more arms 610 may extend from a slider 620 configured to slide along a second rail 630. In operation, at FIG. 6A, the slider 620 may begin at a back end of the second rail 630 and the catheter sheath and guide needle may be connected to one another. Subsequently, as the catheter sheath and guide needle are advanced towards the target vessel, the slider 620 may engage a magnet 640. In some examples, the slider 620 may automatically attach to the magnet 640 as soon as contacts or comes close to the magnet 640, depending on the strength of the magnet 640. In other examples, the slider 620 may not attach to the magnet 640 until the magnet 640 is actuated in response to detection of reaching the target vessel, which may be sensed using any of the example sensing mechanisms described in the other examples of the present disclosure. The magnetic force may exceed the pulling force exerted on the catheter sheath by the guide needle, such that once the slider 620 engages the magnet 640, moving the guide needle backward may cause the guide needle to separate from the catheter sheath.

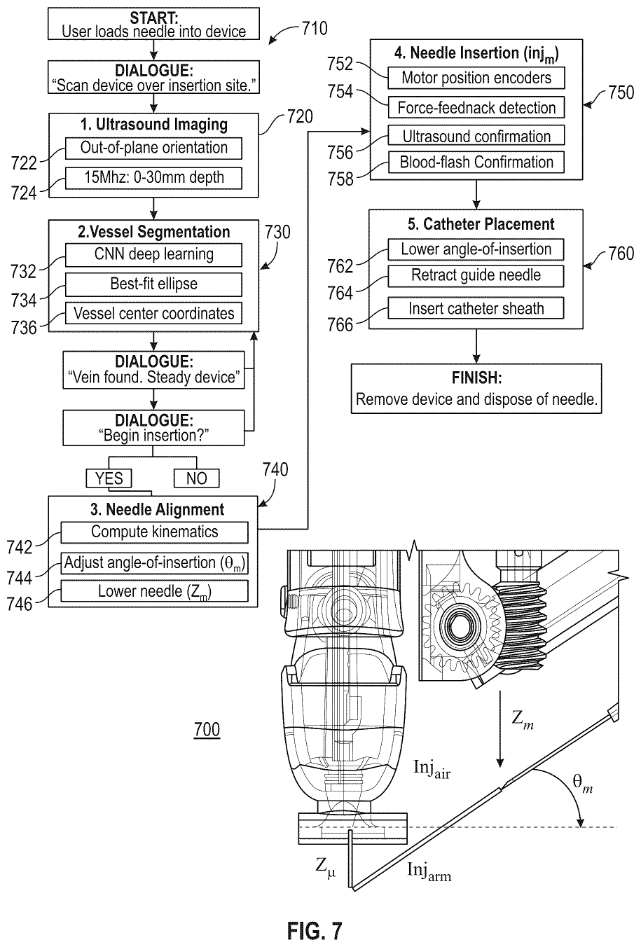

Operation of the mechanical arrangement 300 may be automated along with other aspects of the catheterization device. For example, the carriage may automatically disengage from the catheter sheath support in response to detection of vessel wall puncture. For further example, disengagement may automatically result in retraction of the carriage, either through a mechanical process such as biasing from one or more springs, or through a different automated process such as operation of a motor. Lastly, the catheter may continue to be advanced by an automated program of the catheterization device during retraction of the guide needle and carriage. FIG. 7 illustrates a flow diagram of an example automated workflow 700 of a control device for controlling operation of a catheterization device during a catheterization event. The workflow may begin, after manually loading a needle into the device 710, with a scanning operation, whereby the probe of the device may be positioned over the target vessel. Positioning the device may include collecting image data, such as image frames from a camera or video recording device. The scanning operation may further include ultrasound imaging 720 of the target location using the device probe. The ultrasound imaging 720 may include any one or combination of in-plane imaging, out-of-plane imaging 722 or 3D imaging, and may provide imaging underneath the skin of the patient (e.g., 0-30 mm deep for peripheral venous access, deeper than 30 mm for central venous access) using ultrasonic waves 724. The gathered image(s) may be stored locally or remotely at a remote storage location connected to the device over a network connection. Local and remote storage may be useful for overview or review of the workflow during or after the operations.

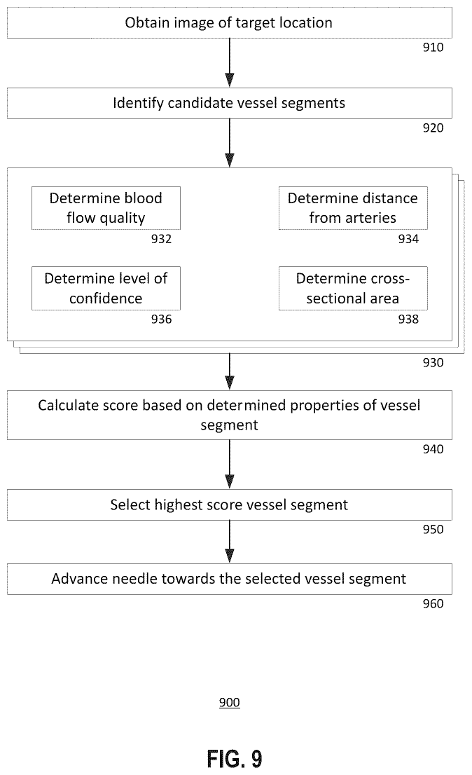

The workflow may continue with image analysis of the gathered image(s). This may involve identifying a target vessel for catheterization. Target vessel identification may itself involve vessel segmentation 730, whereby the obtained images are parsed to identify segments of candidate target vessels and ultimately select a target segment. The segmentation 720 may be performed using a convolutional neural network (CNN) 732, and may involve defining boundaries of each segment by a best-fit ellipse 734 and a center of the target segment according to a vessel segment center coordinate determination 736. In some cases, multiple vessels may be identified and a best vessel may be selected. Further detail about vessel selection is provided hercin in connection with FIG. 9. Ultimately, the chosen vessel may be tracked on a frame-by-frame basis. In the case of a vein, the vein may be further analyzed to determine blood flow through the vessel to ensure that it is a good candidate for the cannulation.

Once the target vessel segment has been selected, a needle alignment process 740 may automatically begin. Optionally, a user input may further act as a determination of whether to begin the cannulation process. If it is decided not to initiate cannulation (NO), such as if the user is not satisfied with the identified target vessel, then operations may revert back to image analysis and vessel segmentation, so that another target vessel segment may be selected. Otherwise, if insertion is to begin (YES), the workflow may continue to robotic control, which may involve computing kinematics of the free-held device if it moves above the target location 742, aligning the cannulation instrument along the Y and Z axes in order to maintain a trajectory towards the target vessel 744, and advancing the cannulation instrument along the depth of insertion axis towards the target vessel 746.

When the catheterization instrument reaches the target vessel, the workflow 700 may also perform a catheter placement process 750 to ensure proper insertion of the catheter into the target vessel. The catheter insertion process 750 may involve lowering an angle of insertion of the guide needle 752, retracting the guide needle 554, and inserting the catheter sheath into the target vessel 756. After completion of the catheter placement, the device may be moved away from the patient and the guide needle may be removed from the device. Some or all steps of the catheter placement process 750 may be monitored using the imaging unit, whereby one or more steps may be confirmed by visualization before proceeding to a next step of the process. Long-axis (longitudinal) orientation of an ultrasound probe may be advantageous for imaging the catheter sheath during insertion.

Lowering the angle of insertion of the guide needle 752 may be performed as part of an automated routine along with insertion of the needle and catheter. One example routine may involve gradually adjusting the angle of insertion of the instrument at different stages of the insertion process. For instance, after aligning the catheter with the target vessel segment, the angle of insertion may be made about 30 degrees. Subsequently, after inserting the catheter into the target vessel center, the angle of insertion may be lowered to about 15 degrees. Finally, as the catheter is slowly inserted into the vessel lumen, the angle of the instrument may be changed to about 10 degrees. This may be performed while or before the guide needle is automatically retracted 754. Additionally, as can be seen from FIG. 7, the degrees of freedom of the device allow for the catheter to be moved in a direction of vertical translation Zₘ, a direction of needle insertion Injₘ, and an angular direction to change the angle of insertion θₘ, thus making the above-described routine possible.

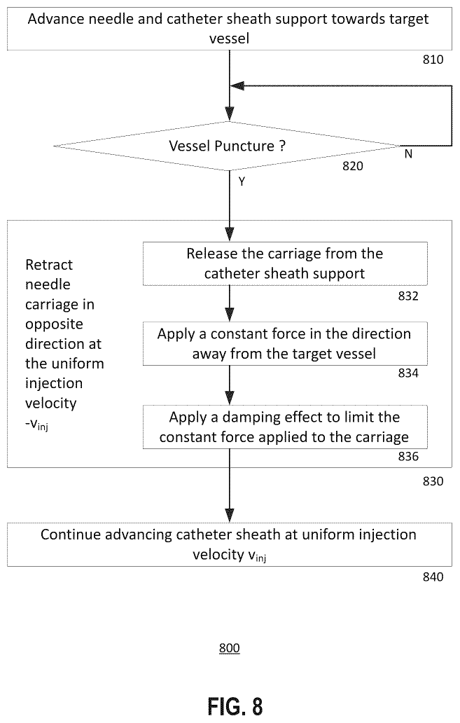

FIG. 8 is a flow diagram of an example routine 800 for the catheter placement process in accordance with the present disclosure. At block 810, the guide needle and catheter sheath are advanced in a direction of injection towards the target vessel location. At this stage, the guide needle and catheter sheath may be locked together using a latching or other connecting element. At block 820, the device continuously or repeatedly checks whether the target vessel has been successfully punctured. Successful puncture may involve an indication of increased pressure, blood flash, or any one or more other indications described herein.

Upon detection of a successful puncture, operations may continue at block 830 with the guide needle and carriage being retracted in a direction opposite the direction of injection. The retraction may be controlled to have a unform velocity, which may be equal and opposite to the velocity at which the guide needle and catheter sheath are advanced towards the target vessel location beginning at block 810.

FIG. 8 shows an example subroutine 800 for retracting the needle carriage and guide needle. In the subroutine, at block 832, the carriage is first released from the catheter sheath support. Then, at block 834, a constant force is applied on the carriage, the constant force pushing or pulling the carriage away from the target vessel location. Additionally, at block 636, a speed or force limiting effect is applied to the constant force to ensure that the velocity at which the carriage retracts away from the catheter heath does not exceed a predetermined amount. The routine 800 further includes, at block 840, the catheter sheath and catheter sheath support continuing to be advanced towards the target vessel as the needle carriage and guide needle are retracted. Since the advancement occurs at the same velocity as the retraction, the guide needle may effectively be kept stationary relative to the target vessel, while the catheter sheath continues to advance into the target vessel. This can prevent the catheter sheath from buckling as the guide needle is being removed.

FIG. 9 shows an example subroutine for selecting a target vessel for the catheter insertion. The target vessel may be a segment of a vessel identified using a vessel segmentation routine. The vessel segmentation routine may use a machine learning model such as a convolutional neural network (CNN) in order to identify candidate segments for catheterization. In one implementation, the machine learning model may be based on the U-Net architecture which provides a pixel-level semantic labeling of each ultrasound image obtained by the imaging unit. Image labels identify which pixels belong or do not belong to vessel segments. The model may be trained and validated using previously collected ultrasound images of blood vessels.

At block 910, the device obtains one or more images of a target location. The target location may be a location under the patient's skin containing one or more potential target vessels for insertion of the catheter. At block 920, the device identifies a plurality of candidate vessel segments.

At block 930, properties or characteristics of each of the candidate vessel segments may be determined. Properties or characteristics of the segments may include but are not limited to, a blood flow quality 932, a distance of the candidate segment from nearby arteries 934, a confidence value indicating a degree or level of confidence of the machine learning model to identify the candidate segment 936, and a cross-sectional area of the candidate segment 938. The blood flow quality measurement 932 may be obtained by block tracking approaches, including but not limited to optical detection or measuring doppler signal strength of blood flowing through the candidate segment. The distance from arteries 934 and cross-sectional area 938 may be determined based on visual data collected by the imaging unit. The confidence value 936 may account for a confidence of the machine learning model to correctly identify the candidate segment. Additional factors such as a depth and angle of the candidate segment relative to the device may be included as properties or characteristics of each of the candidate vessel segments. A respective value may be calculated and assigned for each of the determined properties or characteristics.

At block 940, the device calculates a score for each candidate segment based on the determined characteristics or properties of the segment at block 930. In one example, cach value calculated at block 930 may be weighted by a respective weighting factor and the weighted values may be summed to arrive at a weighted sum. The weighted sum may then represent the overall score for the candidate segment. The candidate segment with the highest score may then be the best candidate for insertion of the catheter. Weightings for each factor may be determined in advance and stored in memory of the device. In general, certain factors such as cross-sectional arca typically have more impact on vessel segment selection and thus can be assigned greater weights than other factors.

In some examples, the score may be influenced by additional factors, such as vessel comprehensibility to indicate a probability of vessel collapse or deformation based on measurements of vessel elasticity, established clinical rules and conventional practices.

At block 950, the highest score candidate vessel segment may be selected for catheterization and the device may initiate a program for, at block 960, advancing the catheter and guide needle towards the selected segment. In this regard, the selection may be fully automated all the way from obtaining the image and other sensor data to the positioning unit initiating advancement of the catheter towards the selected segment.

The above example subroutines may be performed using the processors, microcontrollers and memory included in the catheterization device, such as the components shown and described in connection with FIG. 2. Additionally, it should be recognized that steps of the example subroutines may in some instances be carried out in a different order or simultaneously, that some steps may be omitted from some example routines, or that other steps may be added.

The present disclosure generally describes techniques for catheterization using a fully automated handheld device. However, it should be understood the same or similar techniques may be beneficial and applicable to other devices that are not handheld such as bench-mounted devices, other devices that are not fully automated such as a partially automated or manual catheterization device. For example, the techniques described herein for vessel segmentation and selection is relevant for any device used for catheterization, regardless of how the needle and catheter sheath are advanced and operated under the patient's skin. For further example, the techniques for automatically disengaging a catheter sheath from a needle can be applied to any catheterization device, regardless of whether the sheath is advanced towards its target location in a manual or automated fashion.

The present disclosure generally describes guidance and release of a catheter into the target vessel of a patient. However, it should be recognized that at least some of the above-described devices and techniques may be used to guide or release other instruments into a target vessel of a patient, such as a cannula into a vein of the patient. Additionally, the above-described devices and techniques may be used for other venous or arterial access applications, including but not limited to guide wire threading, venous infusions, short-term peripherally inserted catheters, long-term peripherally inserted catheters, central venous catheters, intravenous fluid resuscitation, introduction of endovenous devices, initiating hemodialysis, arterio-venous fistulas, arterial blood gas sampling, intra-arterial blood pressure measurement, arterial blood transfusion, and introduction of endoarterial devices. The devices and techniques may further be implemented in other needle insertion procedures, including but not limited to peripheral nerve blocks, targeted tissue biopsies, fluid aspiration and drainage, joint aspirations and infusions, needle-based ablations, spinal taps, introducing trocars, and percutaneous surgeries.

The above-described devices and methods are applicable to both human patients and other animal subjects, such as in veterinary applications. More generally, those skilled in the art will readily understand and appreciate that the above-described devices methods for correcting a trajectory of a handheld cannulation instrument based on movement of a target location may be applicable to any cannulation procedure in which there can be an unexpected movement of the subject, an unexpected movement of one holding the device, or both.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An automated cannulation method comprising:
receiving, by one or more processors, imaging data of a target location containing one or more target vessels for insertion of a cannula under a patient's skin;
identifying, by one or more processors, a plurality of candidate vessel segments from among the one or more vessels;
determining, by the one or more processors, a plurality of characteristics of each of the candidate vessel segments based on the imaging data, wherein at least one of the plurality of characteristics is a vessel cross-sectional area;
assigning, by the one or more processors, a respective plurality of values for each of the identified candidate vessel segments based on the determined plurality of characteristics, each value corresponding to a respective characteristic;
calculating, by the one or more processors, a total score for each of the identified candidate vessel segments based on the respective plurality of values;
selecting, by the one or more processors, a highest scoring candidate vessel segment based on the calculated total scores; and
outputting, by the one or more processors, the selected highest scoring candidate vessel segment.

2. The method of claim 1, further comprising assigning a respective predetermined weight to each of the respective plurality of values of the plurality of identified candidate vessel segments,
wherein, for each identified candidate vessel segment, the total score of the identified candidate vessel segment is a weighted sum of its corresponding plurality of values.

3. The method of claim 1, wherein the plurality of characteristics includes a distance of the identified candidate vessel segment from the patient's arteries, wherein the distance of the identified candidate vessel segment from the patient's arteries is determined based on the imaging data.

4. The method of claim 1, wherein the plurality of characteristics includes a quality of blood flow through the identified candidate vessel segment, wherein the quality of blood flow is determined based on doppler signal strength derived from the imaging data.

5. The method of claim 1, wherein identifying the plurality of candidate vessel segments from among the one or more vessels is performed using a machine learning model, and wherein the plurality of characteristics includes a confidence level output by the machine learning model.

6. The method of claim 5, wherein the machine learning model is a convolutional neural network.

7. A method for automated catheterization comprising:
detecting, by a sensor, vessel wall puncture of a target vessel by a needle supporting a catheter sheath; and
in response to detection of the vessel wall puncture:
automatically releasing a carriage of the needle from a catheter sheath support to which the catheter sheath is mounted;
automatically retracting the carriage along an axis of insertion of the needle at a uniform velocity relative to the catheter sheath support; and
automatically advancing the catheter sheath into the target vessel along the axis of insertion of the needle at the uniform velocity while the carriage is automatically retracting such that a position of the needle relative to the target vessel is maintained.

8. The method of claim 7, wherein automatically retracting the carriage comprises applying a constant force to the carriage in a direction away from the target vessel along the axis of insertion of the needle, and wherein the constant force is controlled to not exceed a predetermined threshold.

9. The method of claim 7, wherein the sensor is a force sensor, and wherein automatically releasing the carriage comprises actuating a solenoid latch in response to detection of the vessel wall puncture by the force sensor.

10. The method of claim 7, further comprising receiving an indication of successful catheterization separate from the indication of vessel wall puncture, wherein automatically releasing the carriage in further in response to both the detection of vessel wall puncture and the indication of successful catheterization.

11. The method of claim 10, wherein the indication of successful catheterization is determined from at least one of:
an infrared emitter and collector sensor for detecting blood flash;
a force profile of force data collected at the force sensor during insertion of the needle into the target vessel; or
a temperature reading indicating a presence of blood flash.

12. The method of claim 7, further comprising:
receiving, by one or more processors, imaging data of a target location under a patient's skin;
identifying, by one or more processors, a plurality of candidate vessel segments from among the one or more vessels;
assigning, by the one or more processors, a respective likelihood of success value for each of the identified candidate vessel segments, the likelihood of success value indicating a likelihood of success of canulation using the corresponding candidate vessel segment;
determining, by the one or more processors, a cross-sectional area of each of the candidate vessel segments based on the imaging data;
assigning, by the one or more processors, a respective size value for each of the identified candidate vessel segments;
calculating, by the one or more processors, a total score for each of the identified candidate vessel segments based on the respective likelihood of success values and the respective size values;
selecting, by the one or more processors, a highest scoring candidate vessel segment based on the calculated total scores; and
advancing, by the one or more processors, the guide needle and catheter towards the selected highest scoring candidate vessel segment of the target vessel.

13. A device for automated catheterization comprising:
a catheter sheath support configured to be mounted to a catheterization device, wherein the catheter sheath support is configured to hold a catheter sheath;
a carriage configured to support a guide needle;
a connector configured to removably engage the catheter sheath support to the carriage, wherein the carriage is configured to translate along an axis of insertion of the guide needle independent of the catheter sheath support when the carriage is disengaged from the connector; and
a constant force actuator configured to control separation of the carriage from the catheter sheath support at a uniform velocity in response to disengagement of the connector.

14. The device of claim 13, wherein the constant force actuator is a spring.

15. The device of claim 13, wherein the uniform velocity is equal to a velocity of insertion of the catheter sheath controlled by an injection motor of the handheld catheterization device.

16. The device of claim 13, further comprising a rotary speed limiter configured to limit a magnitude of the uniform velocity to a predetermined threshold.

17. The device of claim 13, further comprising a force sensor positioned in line with the axis of insertion of the guide needle and is configured to:
detect vessel wall puncture; and
transmit a vessel wall puncture signal in response to detection of the vessel wall puncture.

18. The device of claim 17, wherein the connector is configured to disengage in response to the vessel wall puncture signal.

19. The device of claim 18, wherein the connector is a solenoid latch, and wherein the vessel wall puncture signal is an electrical voltage for actuating the solenoid latch.

20. The device of claim 18, further comprising a blood flash sensor configured to detect successful insertion of the guide needle, wherein the connector is configured to disengage further in response to a signal indicating detection of successful insertion of the guide needle by the blood flash sensor.

21. A device for automated catheterization comprising:
a first plurality of prongs extending from a bottom surface of a catheterization device, the first plurality of prongs having an open state and a closed state, wherein the first plurality of prongs are configured to hold a catheter sheath in place relative to the catheterization device when in the closed state;
a track positioned on the bottom surface of the catheterization device;
a second plurality of prongs extending from the track and configured to hold a guide needle;
a motor configured to control the track, wherein control of the track causes the guide needle to move, wherein movement of the guide needle causes the catheter sheath support to move when the first plurality of prongs are in the open state; and
a controller configured to transition the first plurality of prongs from the open state to the closed state in response to detection of successful catheterization.

22. The device of claim 21, wherein successful catheterization is detected based on at least one of:
an infrared emitter and collector sensor for detecting blood flash;
a force profile of force data collected at the force sensor during insertion of the needle into the target vessel; or
a temperature sensor to detecting a temperature increase indicative of blood flash.

23. A device for automated catheterization comprising:
a rail mounted to a bottom surface of a catheterization device;
a first support connected to the rail and configured to slide forward and backward along the rail, wherein the first support is configured to hold a catheter sheath;
a magnet positioned at a front end of the rail, wherein the first support is configured to magnetically attach to the magnet;
a track positioned on the bottom surface of the catheterization device;
a second support extending from the track and configured to hold a guide needle; and
a motor configured to control the track, wherein control of the track causes the second support to move the guide needle, wherein movement of the guide needle causes the catheter sheath support to move and the first support to slide along the rail when the first support is not magnetically attached to the magnet.

24. The device of claim 23, further comprising a controller configured to actuate the magnet in response to detection of successful catheterization, wherein actuation of the magnet is configured to cause the first support to be magnetically attached to the magnet.

25. The device of claim 24, wherein successful catheterization is detected based on at least one of:
an infrared emitter and collector sensor for detecting blood flash;
a force profile of force data collected at the force sensor during insertion of the needle into the target vessel; or
a temperature sensor to detecting a temperature increase indicative of blood flash.
